(19) United States
(12) Patent Application Publication
(10) Pub. No.: US 2010/0105940 A1
(43) Pub. Date: Apr. 29, 2010

(54) FULL CATALYST, PRODUCTION THEREOF, AND USE THEREOF IN AN AMMOXIDATION PROCESS

(75) Inventors: Sabine Huber, Limburgerhof (DE); Randolf Hugo, Dirmstein (DE); Kirsten Dahmen, Mannheim (DE); Thomas Preiss, Weisenheim (DE); Hartmut Hibst, Schriesheim (DE)

Correspondence Address: CONNOLLY BOVE LODGE & HUTZ, LLP, P O BOX 2207, WILMINGTON, DE, 19899, US

(73) Assignee: BASF AKTIENGESELLSCHAFT, Ludwigshafen (DE)

(21) Appl. No.: 11/996,356

(22) PCT Filed: Jul. 12, 2006
(86) PCT No.: PCT/EP2006/064134

§ 371 Date: Jan. 22, 2008

(30) Foreign Application Priority Data
Jul. 20, 2005 (DE) 10 2005 033 825.9

(51) Int. Cl. C07C 253/22; B01J 23/22; B01J 23/18; B01J 23/30

(52) U.S. Cl. 558/311; 502/312

(57) ABSTRACT

Catalysts comprising: (a) a support material comprising a component selected from the group consisting of aluminum oxide, silicon dioxide, aluminum silicate, magnesium silicate, titanium dioxide, zirconium dioxide, thorium dioxide, silicon carbide, and mixtures thereof; and (b) an active material comprising a mixture of vanadium (V) and antimony (Sb) and tungsten (W) and/or molybdenum (Mo), and optionally, at least one alkali metal, wherein the vanadium, antimony, tungsten and/or molybdenum and at least one alkali metal are each present in oxidic form; wherein the support material is provided in a form selected from the group of shapes consisting of spherical or approximately spherical and having a diameter of 2 to 10 mm, tubular or rod-shaped and having a diameter of 1 to 10 mm and a length of 2 to 20 mm, granular having a maximum diameter of 2 to 20 mm, and combinations thereof; and wherein the catalyst is diluted with an inert material; along with processes for preparing such catalysts and their use in preparing nitriles via gas phase ammonoxidation.

The present invention relates to

A full catalyst comprising

a) a support material selected from among aluminum oxide, silicon dioxide, aluminum silicate, magnesium silicate, titanium dioxide, zirconium dioxide, thorium dioxide, silicon carbide and mixtures thereof and

b) vanadium (V) and antimony (Sb) and at least one element selected from among molybdenum (Mo) and tungsten (W), in each case in oxidic form, as active components,

a process for producing this full catalyst and

a process for preparing a monofunctional or polyfunctional isoaromatic or heteroaromatic nitrile by catalytic ammonoxidation of a corresponding isoaromatic or heteroaromatic alkyl compound by means of a gas comprising oxygen and ammonia (ammonoxidation process).

The ammonoxidation of C₁₋₄-alkylisoaromatics and C₁₋₄-alkylheteroaromatics, e.g. toluene, the xylenes or the picolines, is an industrially customary process for the synthesis of the corresponding aromatic nitriles. The reaction is usually carried out in the gas phase using supported catalysts which comprise vanadium together with other elements such as antimony, chromium, molybdenum or phosphorus in oxide form. Supports used are mainly inert metal oxides such as aluminum oxide, silicon dioxide, titanium oxide or zirconium dioxide and mixtures of these oxides.

The strongly exothermic ammonoxidation is customarily carried out in fluidized-bed reactors in industry.

EP-A2-699 476 (BASF AG) relates to supported catalysts which are suitable for ammonoxidation and comprise a) a spherical or approximately spherical support material which consists essentially of aluminum oxide, silicon dioxide, titanium dioxide and/or zirconium dioxide and whose bulk density is from 0.6 to 1.2 kg/l and b) an active composition comprising vanadium and antimony in oxidic form as significant components. These catalysts are suitable for a fluidized-bed process and, according to example 1, have a diameter of about 0.15 mm (determined by the Puralox® aluminum oxide selected).

EP-A1-767 165 (BASF AG) describes a process for preparing aromatic or heteroaromatic nitriles using a supported catalyst which comprises vanadium and consists of from two to thirty particle size fractions having a particular mean diameter and a particular bulk density. These catalysts, too, are particularly suitable for a fluidized-bed process and have, according to example catalyst A, a diameter of about 0.15 mm (determined by the Puralox® aluminum oxide selected).

EP-A2-930 295 (Mitsubishi Gas) teaches an ammonoxidation process for preparing aromatic nitriles over particular V-, Cr- and B-comprising catalysts in a fluidized bed.

STN-Abstract No. 136:19949 of JP-A2-2001 335552 (Showa Denko) relates to the selective partial ammonoxidation of alkylaromatic compounds in the presence of metal oxides comprising vanadium which have been calcined at 400-600° C.

A disadvantage of fluidized-bed processes is the discharge of catalyst (fine catalyst dust because of catalyst attrition) from the fluidization zone of the reactor which is intrinsic to the process and results in the necessity of a cyclone and problems caused by the possible occurrence of catalyst dust in the product.

JP-A-2003 267942 (Mitsubishi Gas) relates to an ammonoxidation process using particular chromium-, vanadium-, molybdenum- and iron-comprising catalysts having aluminum oxide or titanium dioxide as support material which can be used as a fixed bed.

A problem in ammonoxidation processes in a fixed bed is the difficulty of maintaining and controlling the reaction conditions due to the hotspot formation in the fixed bed of catalyst associated with the strongly exothermic reaction. One consequence of this is that the starting material concentration in the feed has to be kept low.

It was an object of the invention to discover an improved economic process for preparing a monofunctional or polyfunctional isoaromatic or heteroaromatic nitrile which overcomes the disadvantages of the present art. The process should be flexible in terms of the setting of the activity of the catalyst, make relatively low reactor temperatures and high starting material concentrations in the reactor feed possible and give the process products in high yields, space-time yields and selectivities. Furthermore, the catalyst used should have a high stability (e.g. measured as lateral compressive strength in newton (N)), a long operating life and a high tolerance toward water.

[Space-time yields are given in “amount of product/(catalyst volume•time)” (kg/(l_{cat.}•h)) and/or “amount of product/(reactor volume•time)” (kg/(l_{reactor}•h))].

We have accordingly found a full catalyst comprising

a) a support material selected from among aluminum oxide, silicon dioxide, aluminum silicate, magnesium silicate, titanium dioxide, zirconium dioxide, thorium dioxide, silicon carbide and mixtures thereof and

b) vanadium (V) and antimony (Sb) and at least one element selected from among molybdenum (Mo) and tungsten (W), in each case in oxidic form, as active components,

wherein the support material is spherical or approximately spherical with a diameter in the range from 2 to 10 mm or tubular or rod-shaped with an (external) diameter in the range from 1 to 10 mm and a length in the range from 2 to 20 mm or granular having a maximum diameter in the range from 2 to 20 mm.

We have also found a process for producing a full catalyst according to any of the preceding claims, wherein the spherical or approximately spherical, tubular, rod-shaped or granular support material is impregnated with a solution or suspension of a vanadium compound and also an antimony compound and a molybdenum and/or tungsten compound and optionally an alkali metal compound, excess liquid is separated off from the resulting mixture and the solid is dried and calcined under oxidizing conditions.

We have also found a process for preparing a monofunctional or polyfunctional isoaromatic or heteroaromatic nitrile by catalytic ammonoxidation of a corresponding isoaromatic or heteroaromatic alkyl compound by means of a gas comprising oxygen and ammonia, wherein such a full catalyst is used as catalyst.

An advantage of the catalyst of the invention is the high activity and mechanical stability.

The spherical or approximately spherical support material preferably has a diameter in the range from 2.5 to 8 mm, in particular from 3 to 7 mm, very particularly preferably from 3.5 to 6 mm, e.g. from 4 to 5 mm.

In the case of tubular (also: hollow-cylindrical) support material, this preferably has an internal diameter in the range from 1 to 7 mm, an external diameter in the range from 2 to 8 mm and a tube length in the range from 2 to 8 mm, in particular an internal diameter in the range from 2 to 6 mm, an external diameter in the range from 3 to 7 mm and a tube length in the range from 3 to 7 mm, very particularly preferably an internal diameter in the range from 3 to 5 mm, an external diameter in the range from 4 to 6 mm and a tube length in the range from 4 to 6 mm.

In the case of rod-shaped support material, this preferably has a diameter in the range from 2 to 5 mm and a length in the range from 5 to 10 mm.

In the case of granular support material, this preferably has a maximum diameter in the range from 3 to 18 mm, particularly preferably in the range from 4 to 16 mm.

The spherical or approximately spherical support material as such is sometimes known and also commercially available (in the case of aluminum oxide, for example the grades from Sasol Germany GmbH).

Suitable spherical or approximately spherical particles preferably have an average shape factor of F>85%. The shape factor is defined as

F=(U₂)²/(U₁)²

where U₁ is the circumference of a particle cross section Q and U₂ is the circumference of a circle having the same cross-sectional area Q. The condition of a minimum shape factor is met when no cross section of the particle corresponds to a smaller value than can be determined statistically.

The tubular support material as such is sometimes known and also commercially available (in the case of aluminum oxide, for example grades having the trade names PU-RALC® and CATAPAL® aluminas from Sasol Germany GmbH).

The spherical or approximately spherical or tubular support material can be produced by subjecting the solution or suspension of an aluminum, silicon, titanium, thorium and/or zirconium compound to spray drying. To produce spherical particles (having, for example, a diameter in the range from 0.1 to 200 μm) by spray drying of solutions of appropriate compounds, suitable compounds are, for example, alkoxides such as ethoxides and isopropoxides, carboxylates such as acetates, sulfates and nitrates, while suitable suspended compounds are hydroxides and hydrated oxides.

In spray drying, the desired particle size and bulk density can be set in a manner known per se.

The particles obtained are converted into the oxides in an oxygen-comprising gas stream at a temperature in the range of, for example, from 500 to 1200° C.

Spheres or tubes having the desired diameters and lengths are subsequently obtained, or obtained after spray drying, by pressing (tableting) and are subsequently calcined/ignited.

In one variant, the particles obtained by spray drying are firstly calcined, then subjected to pressing and then calcined again.

In a further variant, the particles obtained by spray drying are firstly pressed without prior calcination and then calcined.

After pressing to form extrudates (gives rod-shaped support material), these can be crushed (gives granular support material).

The full catalysts of the invention can also be produced by impregnation of the support material.

To produce a full catalysts of the invention, the (if appropriate calcined) support material is impregnated with a solution or suspension of compounds of the metals of the active composition.

On impregnation of the support material, the support is completely impregnated all through. Impregnation of the support material only in the outer region, which would later give a coated catalyst, does not occur.

The intimate mixing of the starting compounds preferably takes place in wet form. The starting compounds are usually mixed with one another in the form of an aqueous solution and/or suspension. Water is preferably used as solvent. The composition obtained in this way is subsequently dried in a manner known per se and calcined under oxidizing conditions, e.g. in a stream of air.

The temperatures used for drying are preferably from 100 to 300° C., and the temperatures in the calcination are from 400 to 750° C., in particular from 450 to 600° C.

Impregnation is preferably carried out using aqueous solutions or suspensions of the compounds of the active catalyst substituents, but any liquids are suitable in principle.

The impregnation solution or suspension is preferably not used in an amount larger than that which can be taken up by the support material, since agglomerates are otherwise obtained during drying and these would firstly have to be broken up again, which could result in formation of particles which do not have the desired spherical or tubular shape. Impregnation can also be carried out in a plurality of steps with intermediate drying between the steps.

Impregnation of the support material is preferably carried out using the active components in the form of aqueous solutions of their salts, in particular salts of organic acids which decompose without leaving a residue during the oxidative calcination. Preference is here given to the oxalates, particularly in the case of vanadium, and the tartrates and acetates, particularly in the case of antimony, with the tartrates also being able to be present in the form of mixed salts, e.g. with ammonium ions. To produce such solutions, the metal oxides can be dissolved in the acids.

The vanadium compounds used can also be a nitrate or vanadate.

The antimony compound used can also be an antimonate.

Molybdenum and tungsten are each preferably used in the form of complexes with tartaric acid, oxalic acid or citric acid or in the form of a molybdate or tungstate.

Metallic W and/or Mo can be oxidized and brought into solution by means of H₂O₂.

Shaping of the full catalysts can be carried out before or after the thermal treatment is carried out.

For example, a full catalysts can be produced from the powder form of the multielement oxide active composition according to the invention or its not yet thermally treated precursor composition (the intimate dry mixture) by compaction to give the desired catalyst geometry (sphere, tube, rod; e.g. by tableting, screw extrusion or ram extrusion), with diluents such as SiO₂, auxiliaries such as graphite or stearic acid as lubricants and/or shaping aids and reinforcing materials such as microfibers composed of glass, asbestos, silicon carbide or potassium titanate optionally being able to be added.

The calcination atmosphere can be realized in a simple fashion by, for example, carrying out the calcination in a furnace through which an O₂-comprising gas mixture, e.g. air, is passed. The calcination temperature is preferably in the range from 400 to 750° C.

The amount of vanadium, calculated as metal, in the catalyst is preferably from 0.5 to 50% by weight, particularly preferably from 0.7 to 10% by weight, more preferably from 1.0 to 7% by weight, especially from 1.5 to 6% by weight,

and the amount of antimony, likewise calculated as metal, is preferably from 0.5 to 50% by weight, particularly preferably from 1 to 20% by weight, more preferably from 2 to 10% by weight.

In a preferred embodiment, the catalysts preferably further comprise from 0.01 to 5.0% by weight, in particular from 0.1 to 3% by weight, e.g. from 0.15 to 2% by weight, of alkali metal, i.e. Li, Na, K, Rb and/or Cs, preferably cesium and/or rubidium, in each case calculated as metal.

The catalysts preferably further comprise from 0.05 to 12% by weight, in particular from 0.1 to 3% by weight, more preferably from 0.01 to 2.5% by weight, of Mo and/or W, in each case calculated as metal.

These amounts are in each case based on the total mass of the catalyst.

Preferred catalysts comprise, based on the mass of the a full catalyst, from 1 to 50% by weight, in particular from 5 to 25% by weight, very particularly preferably from 7 to 20% by weight, of the active components.

In addition, the catalyst can comprise further active components, e.g. compounds of titanium, iron, cobalt, nickel, manganese and/or copper.

In a particular embodiment of the catalyst of the invention, the catalyst comprises no iron (Fe), no chromium (Cr) and/or no boron (B), in each case in oxidic form.

A particularly preferred catalyst according to the invention is a full catalyst comprising

a) aluminum oxide as a spherical or approximately spherical support material having a diameter in the range from 2 to 10 mm, in particular from 4 to 6 mm, and

b) an active composition comprising vanadium (V) and antimony (Sb) and tungsten (W) and cesium (Cs), in each case in oxidic form, and no chromium (Cr) and no iron (Fe).

The catalysts of the invention are suitable for the inventive ammonoxidation reactions in a fixed bed.

Preferred fixed-bed reactors are tube reactors and shell-and-tube reactors as are described, for example, in Ullmann's Encyclopedia of Industrial Chemistry, 6th Ed., keyword “fixed bed reactors”.

The fixed bed of catalyst is located in the metal tubes of the shell-and-tube reactor and the heat transfer medium or media is/are passed around the metal tubes (in the case of more than one temperature zone, a corresponding number of physically separate heat transfer media are passed around the metal tubes). The heat transfer medium is preferably a salt melt. The reaction mixture is passed through the catalyst tubes.

The catalyst tubes are usually made of ferritic steel and typically have a wall thickness of from 1 to 3 mm. Their internal diameter is preferably from 12 to 30 mm, frequently from 14 to 26 mm. Their length is advantageously from 3 to 6 m.

For process engineering reasons, the number of catalyst tubes accommodated in the shell of the shell-and-tube reactor is advantageously at least 5000. The number of catalyst tubes accommodated in the reactor shell is frequently from 10 000 to 30 000. Shell-and-tube reactors having more than 40 000 catalyst tubes tend to be the exception. Within the shell, the catalyst tubes are normally distributed homogeneously (preferably 6 equidistant neighboring tubes per catalyst tube), with the distribution advantageously being selected so that the distance between the central axes of nearest-neighbour catalyst tubes (the catalyst tube spacing) is from 35 to 45 mm (cf., for example, EP-A-468 290).

As heat transfer media, it is particularly advantageous to use melts of salts such as potassium nitrate, potassium nitrite, sodium nitrite and/or sodium nitrate, or of low-melting metals such as sodium, mercury or alloys of various metals.

The full catalyst is preferably diluted with an inert material in the reactor, which enables the activity of the catalyst to be set in a targeted manner.

The inert material can be, for example, steatite spheres, steatite tubes, aluminum oxide spheres, aluminum oxide tubes, silicon dioxide spheres and/or silicon dioxide tubes. The inert material is preferably identical to the support material of the full catalyst used.

The inert material preferably has a geometry (diameter, length) which is similar to or identical with that of the support material of the full catalyst used.

In particular, a dilution profile over the length of the reactor is set by dilution of the catalyst with the inert material. For example, a plurality of zones (e.g. 2, 3 or 4 zones which are formed, for example, by equal distribution of the total catalyst volume) having differing dilution can be advantageously produced.

It is particularly advantageous for the zone at the reactor inlet to have a higher dilution than at the end of the reactor. For example, it is possible to form two zones in which the catalyst in the zone at the reactor inlet is diluted with from 10 to 90% by weight, preferably from 20 to 50% by weight, of inert material and the catalyst in the zone at the end of the reactor is diluted with from 0 to 90% by weight, preferably from 1 to 30% by weight, of inert material. The percentages by weight are in each case based on the total weight of a full catalyst and inert material used in the respective zone.

The height of the inert preliminary bed in the reactor tube is preferably in the range 5-100 cm, and that of the after-bed is preferably in the range 0-100 cm.

The preliminary bed serves to heat the reaction gas in the space upstream of the reaction, while the after-bed serves to hold back abbraded catalyst and prevent it from getting into the subsequent reaction stages.

The inert beds also prevent the catalyst from lifting and moving should a pressure pulse occur; voids and dead volumes are also avoided.

The inert beds also prevent the catalyst from lifting and moving should a pressure pulse occur; voids and dead volumes are also avoided.

The catalysts of the invention are advantageously employed for preparing monofunctional and polyfunctional isoaromatic and heteroaromatic nitriles from the corresponding alkyl compounds (starting materials), e.g. C₁₋₄-alkyl compounds, in particular the methyl compounds.

The amminoxidation according to the invention is of particular importance for the prepartition of o-phthalodinitrile (OPDN) from o-xylene, of isophthaloniditrile (IPDN) from m-xylene, of terephthaloniditrile from p-xylene, of benzonitrile from toluene and of nicotinonitrile from beta-picoline.

In the case of the xylenes, the ammonoxidation of the first methyl group proceeds more quickly than that of the second, so that partial ammonoxidation products can also be obtained easily, e.g. p-methylbenzonitrile from p-xylene.

The aromatic starting materials can bear substituents which are inert under the conditions of the ammonoxidation, i.e., for example, halogen or the trifluoromethyl, nitro, amino or cyano group. Substituents which are not inert are also possible if they are converted into desired substituents under the conditions of the ammonoxidation, for example the aminomethyl group or the hydroxymethyl group.

The ammonoxidation process of the invention is preferably carried out at a temperature in the range from 300 to 550° C., in particular from 350 to 500° C., very particularly preferably from 380 to 490° C., e.g. from 420 to 480° C.

The organic starting compound to be oxidized is preferably taken up in a gas stream comprising ammonia and an oxygen-comprising gas such as air, with the concentration of the starting compound in the gas stream advantageously being set to from 0.1 to 10% by volume, preferably from 0.1 to 5% by volume.

The oxygen content of the gas used for the ammonoxidation is preferably in the range from 0.1 to 25% by volume, in particular in the range from 3 to 15% by volume.

The catalysts of the invention allow a space velocity over the full catalyst in the range from 0.1 to 2 kg of the starting compound per kg of catalyst and per hour.

Unreacted ammonia is advantageously recirculated to the reaction.

Tolunitrile formed in the ammonoxidation of xylene to the corresponding phthalonitrile is advantageously recirculated to the reaction after it has been separated off from the reaction product.

### EXAMPLES

### Example 1

### Production of a Full Catalyst According to the Invention, V₄Sb_{3,1}W_{0,66}Cs_{0,74}Ox

In an 8 l stirred vessel, 1350 g of ice, 1350 g of water and 544.2 g of Perhydrol (from Merck Eurolab, 64271 Darmstadt; 30% strength aqueous solution of H₂O₂ in water; 4.8 mol of H₂O₂) were mixed with stirring. A total of 90.01 g of divanadium pentoxide (from GfE Gesellschaft für Elektrometailurgie, D-90431 Nürnberg; 99.97% by weight of V₂O₅; 1.0 mol of V) were added a little at a time to this cold mixture over a period of 75 minutes while continuing to stir, forming a clear red solution A.

396.8 g of Perhydrol (from Merck Eurolab, 64271 Darmstadt; 30% strength by weight solution of H₂O₂ in water; 3.5 mol of H₂O₂) were placed in a 2 l vessel and a total of 30.35 g of tungsten powder (from Chempur, Feinchemikalien and Forschungsbedarf GmbH, 76204 Darmstadt; 99.95% by weight of W; 0.165 mol of W) were added thereto a little at a time over a period of 60 minutes while stirring to give a clear solution B.

In a 500 ml vessel, 35.58 g of cesium acetate (from Chemetall, D-60323 Frankfurt; 99.8% by weight of CsOAc); 0.185 mol of Cs) were dissolved in 100 ml of water to give a clear solution C.

The solution B was subsequently added to the solution A while stirring. While continuing to stir, 113.7 g of diantimony trioxide (from Antraco, D-10247 Berlin; 99.35% by weight of Sb₂O₃; 0.775 mol of Sb) and 255.1 g of Perhydrol (from Merck Eurolab, 64271 Darmstadt; 30% strength by weight solution of H₂O₂ in water; 2.25 mol of H₂O₂) were added to the resulting clear solution.

The mixture obtained was heated to 90° C. and heated at this temperature for 2 hours. The mixture obtained was subsequently added to the solution C and heated at 90° C. for a further one hour while stirring.

After cooling, the suspension obtained was dried in a spray dryer (Minor, model Hi-Tec, from Niro GmbH, D-75105 Karlsruhe) (inlet temperature=320° C., outlet temperature=110° C.). The black powder obtained had a BET surface area of 165 m²/g. The X-ray powder diffraction pattern of the black powder obtained corresponded to the crystal structure of tetragonal Sb_{0.958}V_{0.959}O₄. 243.5 g of the black powder obtained were mixed dry with 300 g of Pural SB (from Sasol, D-20537 Hamburg; hydrated aluminum oxide having an Al₂O₃ content of 75% by weight) in a laboratory mixer (from Robert Bosch Hausgeräte GmbH, model Bosch Universal 6012, D-81739 München) for 45 minutes. The powder mixture obtained was subsequently kneaded with addition of an aqueous solution of 16.3 g of formic acid (from Merck Eurolab, 64271 Darmstadt; >98% by weight of HCOOH) in 100 ml of water for 15 minutes in a kneader (from Werner & Pfieiderer, D-70469 Stuttgart; model LVK 1.0 K2T) cooled to 16° C. About 50-200 ml of additional water were subsequently added and the mixture was kneaded for 45 minutes while continuing to cool the kneader to give a firm dough. The precise amount of water added depended on the way in which the kneading process proceeded, since the mixture heats up during kneading (up to about 40° C.) and a differing amount of water evaporates depending on the temperature reached. In this case, the amount of added water is selected so that a firm, extrudable dough is obtained after the kneading process. This dough was subsequently transferred to an extruder (from Werner & Pfleiderer, D-70469 Stuttgart; extrusion die having 2 mm holes) and extruded to give round rods having a diameter of 2 mm. The extrudates obtained were dried overnight at 120° C. in air and broken up to give granules having a particle size of 2-3 mm.

### Example 2

### Preparation of Isophthaloniditrile (IPDN) by Ammonoxidation of Metaxylene

The catalyst granules from example 1 diluted with 90% by weight of 2-3 mm steatite spheres were installed in dilute form in a fixed-bed reactor having an internal diameter of 16 mm and a bed length of the catalyst of 60 cm.

A gas mixture comprising 1% by volume of m-xylene, 9% by volume of ammonia and 12% by volume of oxygen (balance to 100% by volume: nitrogen) was passed over the catalyst at a reactor temperature of 430° C.

At a conversion of m-xylene of 82%, selectivities to IPDN of 62% and to tolunitrile of 26% were obtained.

1-30. (canceled)

31. A catalyst comprising:
(a) a support material comprising a component selected from the group consisting of aluminum oxide, silicon dioxide, aluminum silicate, magnesium silicate, titanium dioxide, zirconium dioxide, thorium dioxide, silicon carbide, and mixtures thereof; and
(b) an active material comprising a mixture, the mixture consisting of vanadium (V) and antimony (Sb) and tungsten (W) and at least one alkali metal, wherein the vanadium, antimony, tungsten and at least one alkali metal are each present in oxidic form;
wherein the support material is provided in a form selected from the group of shapes consisting of spherical or approximately spherical and having a diameter of 2 to 10 mm, tubular or rod-shaped and having a diameter of 1 to 10 mm and a length of 2 to 20 mm, granular having a maximum diameter of 2 to 20 mm, and combinations thereof; and wherein the catalyst is diluted with an inert material.

32. The catalyst according to claim 31, wherein the support material is provided in a form selected from the group of shapes consisting of spherical or approximately spherical and having a diameter of 2.5 to 8 mm, tubular or rod-shaped and having a diameter of 2 to 8 mm and a length of 3 to 18 mm, granular having a maximum diameter of 3 to 18 mm, and combinations thereof.

33. The catalyst according to claim 31, wherein the support material is provided in a tubular form having an internal diameter of 1 to 7 mm, an external diameter of 2 to 8 mm and a tube length of 2 to 8 mm.

34. The catalyst according to claim 31, wherein the support material is provided in a tubular form having an internal diameter of 2 to 6 mm, an external diameter of 3 to 7 mm and a tube length of 3 to 7 mm.

35. The catalyst according to claim 31, wherein the at least one alkali metal is selected from cesium (Cs), rubidium (Rb), and mixtures thereof.

36. The catalyst according to claim 31, wherein the vanadium and antimony are each present in an amount of 0.5 to 50% by weight, calculated as metal and based on the catalyst.

37. The catalyst according to claim 35, wherein the at least one alkali metal is present in an amount of 0.01 to 5% by weight, calculated as metal and based on the catalyst.

38. The catalyst according to claim 31, wherein the tungsten is present in an amount of 0.05 to 12% by weight, calculated as metal and based on the catalyst.

39. The catalyst according to claim 31, wherein the active material is present in an amount of 1 to 50% by weight based on the catalyst.

40. A process for preparing a catalyst, the process comprising:
(a) providing a support material in a form selected from the group of shapes consisting of spherical or approximately spherical and having a diameter of 2 to 10 mm, tubular or rod-shaped and having a diameter of 1 to 10 mm and a length of 2 to 20 mm, granular having a maximum diameter of 2 to 20 mm, and combinations thereof, and wherein the support material comprises a component selected from the group consisting of aluminum oxide, silicon dioxide, aluminum silicate, magnesium silicate, titanium dioxide, zirconium dioxide, thorium dioxide, silicon carbide, and mixtures thereof;
(b) impregnating the support material with a solution or suspension comprising a vanadium compound, an antimony compound and a compound selected from tungsten compounds, molybdenum compounds and mixtures thereof; and
(c) drying and calcining under oxidizing conditions the impregnated support material.

41. The process according to claim 40, wherein the vanadium compound is selected from the group consisting of an oxide, a tartrate, an oxalate, an acetate, a nitrate, a vanadate, and mixtures thereof.

42. The process according to claim 40, wherein the antimony compound is selected from the group consisting of an oxide, a tartrate, an oxalate, an acetate, a antimonate, and mixtures thereof.

43. The process according to claim 40, wherein the solution or suspension comprises a tungsten compound selected from the group consisting of an oxide, a tungstate, and mixtures thereof.

44. The process according to claim 40, wherein the solution or suspension further comprises at least one alkali metal selected from the group consisting of oxides, nitrates, carbonates, hydroxides, and mixtures thereof.

45. The process according to claim 40, wherein the calcining is carried out in the presence of oxygen at a temperature of 400 to 750° C.

46. A process for preparing a nitrile, the process comprising:
(a) providing an isoaromatic or heteroaromatic alkyl compound; and
(h) ammonoxidating the alkyl compound in a reactor with a gas comprising oxygen and ammonia in the presence of a catalyst according to claim 31.

47. The process according to claim 46, wherein ammonoxidating is carried out at a temperature of 300 to 550° C.

48. The process according to claim 46, wherein oxygen is present in the gas in an amount of 0.1 to 25% by volume.

49. The process according to claim 46, wherein the catalyst is arranged in the reactor as a fixed bed.

50. The process according to claim 46, wherein the catalyst is diluted with an inert material.
